(19) United States
(12) Patent Application Publication
(10) Pub. No.: US 2006/0064009 A1
(43) Pub. Date: Mar. 23, 2006

(54) Vessel imaging devices and methods

(76) Inventors: William E. Webler, Escondido, CA (US); Mina Chow, Campbell, CA (US); Jessica G. Chiu, Belmont, CA (US); Dagmar B. Beyerlein, San Francisco, CA (US); Daniel L. Cox, Palo Alto, CA (US); Richard Van Calfee, Houston, TX (US); Jeong Lee, Diamond Bar, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/947,615

(22) Filed: Sep. 21, 2004

(51) Int. Cl. A61M 25/00

(52) U.S. Cl. 600/434

(57) ABSTRACT

Various devices and methods for improving intravascular imaging are disclosed. In one embodiment, fluid dispersion devices are included on a catheter to improve dispersal of a flush solution within a flow of fluid (e.g., blood) in a vessel. In other embodiments, a catheter includes at least one inflatable balloon to selectively partially occlude a vessel to be imaged and/or treated in order to minimize the refractory effects of blood on the imaging/treatment process. In one embodiment, a catheter may image/treat at least a portion of a vessel by moving an imaging/treatment device in a distal direction relative to a proximal section of the catheter.

### FIELD

Imaging or treatment devices including catheters having imaging or treatment capabilities.

### BACKGROUND

There are several methods of imaging the inner walls of various vessels within the body. For example, angioscopy, optical coherence tomography (“OCT”), and intravascular ultrasound (“IVUS”) may all be used to obtain intravascular images. In addition, photodynamic therapy may be administered within a vessel to treat various conditions. For example, light (e.g., blue light and/or ultraviolet light) may be used to destroy (e.g., cell lysis) or treat various target tissues such as tumors and atheromas, including thin capped fibroathroma (“TCFA”) or vulnerable plaque. Each of these imaging and therapy techniques either require or benefit from the elimination of blood within the imaging field/therapy administration area.

An IVUS catheter typically includes an elongated member and an ultrasound transducer located at the distal end or a distal portion of the elongated member. The elongated member is inserted into a blood vessel, and the ultrasound transducer is positioned at a desired location within the blood vessel. An ultrasound transducer typically transmits a specific resonant frequency when it is excited by a pulse. The excited pulse signal causes the ultrasound transducer to emit ultrasound wave(s) in the blood vessel. A portion of the emitted ultrasound wave(s) is reflected back to the ultrasound transducer at tissue boundaries in the blood vessel and the surrounding tissue. The reflected ultrasound waves induce an echo signals in the ultrasound transducer. The echo signals are transmitted to an ultrasound console, which typically includes an ultrasound image processor and possibly a display. The ultrasound console uses the received echo signals to create a depth image the blood vessel and the surrounding tissue. The amplitude of the echo signals determines the image brightness and the time that the echo signals are received after the excited pulse is emitted determines the depth into the tissue that the reflected ultrasound waves came from. Assembling the brightnesses and depths of the reflected ultrasound waves from the echo signals on a display forms the depth image of the tissue.

To produce a radial cross-section image of a blood vessel and the surrounding tissue using IVUS, the ultrasound transducer may be rotated along the axis of the elongated member. Alternatively, the ultrasound transducer may be mounted in an assembly along with a mirror or mirrors. The transducer emits ultrasonic energy in a substantially axial direction and the mirror or mirrors is/are oriented to deflect the emitted ultrasonic energy in a radial direction.

OCT is analogous to ultrasound imaging but measures the intensity of back-scattered infrared light rather than ultrasound. To image a blood vessel and/or surrounding tissue of a patient using OCT, an optical fiber (e.g., a fiber having an outside diameter on the order of 100-150 microns) is inserted into a blood vessel and light is transmitted through the optical fiber and emitted at a distal end into the blood vessel. The light is typically produced by a laser, e.g., a laser diode and split into two parts. One part is sent into the optical fiber in the patient and the other part, called the reference beam, is sent to an interferometer or detector via a controlled path length. The light reflected back from the tissue is transmitted through the optical fiber to the interferometer or detector, which compares the reflected light from the tissue to the reference beam to obtain the intensity of the light reflected back from the tissue at the same path length as that of the reference beam.

By varying the path length of the reference beam, the intensities of the light reflected by the tissues at different depths into the tissue may be detected and assembled into a depth image of the tissue. In addition, the OCT system may include a motor unit for providing drive torque to the optical fiber to rotate the optical fiber during imaging. This enables a radial cross-sectional image of the inside of the blood vessel and/or surrounding tissue to be obtained.

Theoretically, OCT should be able to image about 2.5 millimeters (mm) to 3 mm into blood or tissue. Those that make/experiment with OCT imaging systems have difficulty imaging through more than approximately 2 mm of blood or vessel tissue and often report results of imaging 1.2 to 1.7 mm into blood or vessel tissue. This is likely due to the fact that the light used in OCT imaging systems generally has a wavelength short enough to interact with individual red blood cells (and other small tissue structures) and this interaction can be quite complex/difficult to model. Use of longer wavelengths to avoid red blood cell interaction results in a loss of depth resolution for the detection of, for example, vulnerable plaque.

Red blood cells have a slightly higher index of refraction than the plasma in which they are suspended and are shaped like concave lenses so that the OCT light may be redirected and refocused as the light passes through each red blood cell. Thus, it is desirable to minimize the effect of the blood's interference with the light from the imaging system as it propagates through the vessel towards the vessel wall, into the vessel wall and is reflected back to the device.

One area of particular interest in cardiovascular research is identifying vulnerable plaque or plaques that may be in danger of becoming a vulnerable plaque. A vulnerable plaque generally has a thin cap that is 0.05 mm to 0.10 mm thick or thinner that covers a core filled with lipids, white cells and necrotic by-products (cell debris). Imaging into a vessel wall to a depth on the order of about 0.25 mm should be adequate to detect a vulnerable plaque or a plaque that may be in danger of becoming a vulnerable plaque. A typical OCT system will have a resolution of about 0.025 mm or smaller. Thus, OCT will show the true thickness of a vulnerable plaque's cap, at least well enough to identify the plaque as a vulnerable plaque. Current IVUS systems, on the other hand, have a resolution of about 0.15 mm. Current IVUS systems are capable of imaging pre-vulnerable plaques, but may not be able to image the thickness of a vulnerable plaque's cap—any cap will appear at least 0.15 mm thick.

Various techniques and devices have been used to flush blood from the imaging field area/therapy administration area with limited success. For example, flushing a coronary artery to remove blood from the field of view is normally accomplished by injecting saline into the vessel to be imaged, either through a guide catheter or a catheter/sheath that surrounds/incorporates the imaging device. However, this technique has several drawbacks.

First, when enough saline solution or other isotonic biocompatible water-based solution is introduced to replace or dilute the blood, the amount of oxygen in the solution is very small in comparison to the amount of oxygen contained in the blood. Thus, the time window for imaging is limited by the ischemic consequences of the solution on the heart muscle (e.g., reduction in blood flow). The longer the duration of the flush, the more severe the consequences are to the heart muscle. Since imaging is generally desired in patients usually already suffering from ischemia or previous cardiac muscle ischemic tissue damage, the safe/pain-free imaging time period is short.

Second, blood flow in coronary arteries is laminar and generally tends to flow in streamlines, not mixing very rapidly with adjacent streamlines. Thus, injected solutions tend to flow in their own streamlines, leaving some areas of blood flow not completely displaced/mixed or leaving eddies of blood at branch points or at areas protected/created by the presence of the imaging device.

Third, most water-based flushing solutions have a viscosity that is significantly less than that of blood. Thus, the flow rate of the flush must exceed the normal flow rate of the blood in the vessel in order to create enough pressure in the vessel to exceed the blood pressure and displace the blood. In other words, the resistance to flow in the vessel is lower for the flush than for the blood.

As the flush replaces the flowing blood, an ever-increasing flow rate of the flush is required. For example, the decreased resistance of the flush requires more overall fluid (e.g., flush) to maintain the natural flow rate. Moreover, the vessel will dilate in response to the ischemic properties caused by an increased amount of oxygen deficient fluid in the vessel. Thus, the flush flow rate must be increased until a peak flow rate is reached, wherein the flush effectively completely replaces the blood in the artery. The volume of flush required to achieve this peak flow rate can be quite high during extended imaging periods, like those commonly used with IVUS.

Fourth, in most injection configurations, the required high flush flow rate enters the artery via a relatively small flow cross section, resulting in a very high injection velocity. This may create high velocity jets of flush, which can damage vessel walls. Additionally, the pressures and volumes required are not easily accomplished by manual injection. Therefore, an automated injection device is desirable.

Alternatively, injection of a fluid more viscous than saline (e.g., a contrast agent) may utilize a lower flow rate, but the catheter injection pressure is relatively unchanged due to the higher viscosity. A high viscosity flush also increases the time required to wash out the flush (e.g., longer ischemia time). Moreover, contrast agents are quite expensive relative to normal flushing solutions.

Several methods to deal with these problems of a typical flush have been utilized in the past. For example, oxygenated blood can be withdrawn from the patient, and certain materials may be added to the blood to increase the index of refraction of its plasma to match that of the red blood cells. This oxygenated blood, with a higher index of refraction of its plasma, can then be used as the flush. Alternatively, the materials to increase the index of refraction of the plasma may be added systemically without withdrawing any blood from the patient.

In either case, such a procedure would eliminate/effectively minimize the lens effect and the reflection effect of the red blood cells. Since the red blood cells are oxygenated, ischemia is not a problem. It has been reported that contrast can be used to make this index of refraction change to the plasma.

Changing the index of refraction on a systemic level is very difficult and can be toxic. It is easier and faster to perform the index of refraction change with blood withdrawn from the body. However, changing the index of refraction outside of the patient's body requires extra equipment and a time-consuming index matching procedure and introduces issues involving increased blood exposure (e.g., to the environment). Moreover, the streamline and injection problems discussed above would still be a challenge, and hemolysis (e.g., the destruction or dissolution of red blood cells, with subsequent release of hemoglobin) could be an added issue to consider.

### SUMMARY

Various devices and methods of improving vessel imaging and photodynamic therapy are disclosed. In one embodiment, a flush may be introduced into a flow of fluid (e.g., blood) within a vessel in order to minimize the amount of blood present in an imaging field or photodynamic therapy administration area of an imaging/therapy device. In order to improve mixing with the blood, the flush may be dispersed or mixed with the blood by a fluid dispersion device connected to a catheter adjacent to the lumen opening from which the flush is introduced into the vessel.

In other embodiments, a catheter may be inserted into a vessel to be imaged and/or treated, wherein the catheter includes at least one balloon to selectively partially occlude the vessel so that blood is channeled and/or redirected to enable imaging and/or treatment. Such a device enables imaging and treatment while minimizing the potential ischemic effects of cutting off blood flow during imaging or treatment (e.g., by introducing too much flush during the procedure).

In one embodiment, a catheter is inserted into a vessel to be imaged or treated, and the imaging device images (or the photodynamic therapy device emits light) by moving in a distal direction relative to a proximal section of the catheter.

In various embodiments, a timer may be used to time the introduction of a flush into a flow of fluid based on a cardiac cycle of a subject (e.g., a patient). Timing may include, for example, determining an appropriate time to begin introducing a flush during a cardiac cycle, determining the appropriate duration of flush introduction, and/or introducing the flush, taking into account flow rate and distance between a lumen opening and an imaging or treatment field of a device, at a time and for a duration to maximize the amount of time that the flush will be in the imaging field/treatment area of a device during a portion of the cardiac cycle.

### DESCRIPTION OF THE DRAWINGS

Various embodiments are illustrated by way of example and not by way of limitation in the figures of the accompanying drawings in which like references indicate similar elements. It should be noted that references to “an,” “one,” “the,” “other,” “another,” “alternative,” or “various” embodiments in this disclosure are not necessarily to the same embodiment, and such references mean at least one.

FIG. 1 shows a side view of a catheter assembly with one embodiment of a fluid dispersion device coupled to the catheter.

FIG. 2 shows a cross-sectional view of the catheter of FIG. 1 through line 1-1′.

FIG. 3 shows a side view of a distal end of a primary cannula having a protrusion formed thereon.

FIG. 4 shows a cross-sectional view of the cannula of FIG. 3 through line 3-3′.

FIG. 5 shows another embodiment of a distal end of a primary cannula having a protrusion formed thereon.

FIG. 6 shows a cross-sectional view of the cannula of FIG. 3 through line 5-5′.

FIG. 7 shows another embodiment of a distal end of a primary cannula having a protrusion formed thereon.

FIG. 8 shows a cross-sectional view of the cannula of FIG. 3 through line 7-7′.

FIG. 9 shows another embodiment of a distal end of a primary cannula having a protrusion formed thereon.

FIG. 10 shows a cross-sectional view of the cannula of FIG. 3 through line 9-9′.

FIG. 11 shows another embodiment of a distal end of a primary cannula having a protrusion formed thereon.

FIG. 12 shows a cross-sectional view of the cannula of FIG. 3 through line 11-11′.

FIG. 13 shows another embodiment of a distal end of a primary cannula having a protrusion formed thereon.

FIG. 14 shows a cross-sectional view of the cannula of FIG. 3 through line 13-13′.

FIG. 15 shows another embodiment of a distal portion of a primary cannula having multiple protrusions thereon.

FIG. 16 shows a side view of an embodiment of a catheter assembly having multiple flush solution ports.

FIG. 17 shows a side view of a distal portion of a catheter assembly having a flush forward configuration.

FIG. 18 shows a cross-sectional view of the catheter assembly of FIG. 17 through line 17-17′.

FIG. 19 shows a side view of a distal portion of a catheter assembly having a flush forward configuration and an inflatable balloon.

FIG. 20 shows a cross-sectional view of the catheter of FIG. 19 through line 19A-19A′

FIG. 21 shows a cross-sectional view of the catheter assembly of FIG. 19 through line 19B-19B′.

FIG. 22 shows a side view of a distal portion of a catheter assembly having a fluid dispersion device and a flush forward configuration.

FIG. 23 shows a cross-sectional view of the catheter of FIG. 22 through line 22A-22A′.

FIG. 24 shows a cross-sectional view of the catheter assembly of FIG. 22 through line 22B-22B′.

FIG. 25 shows a side view of a distal portion of an embodiment of a catheter assembly within a blood vessel.

FIG. 26 shows a cross-sectional view of the catheter assembly of FIG. 25 through line 25-25′.

FIG. 27 shows another embodiment of a cross-sectional side view of a catheter assembly such as FIG. 25 through line 25-25′.

FIG. 28 shows a side view of a distal portion of a catheter assembly within a blood vessel.

FIG. 29 shows a side view of a distal portion of an embodiment of a catheter assembly within a blood vessel.

FIG. 30 a the cross-sectional side view of the catheter assembly of FIG. 29 through line 29-29′.

FIG. 31 shows a flow chart describing an imaging/treating process of a blood vessel.

FIG. 32 shows a side view of a portion of a catheter assembly having an imaging/treatment device aligned with a bolus in the blood vessel.

FIG. 33 shows the catheter assembly of FIG. 32 at a later point in time.

FIG. 34 shows the catheter assembly of FIG. 33 at a later point in time.

FIG. 35 shows a side view of a distal portion of a catheter assembly.

FIG. 36 shows a cross-sectional side view of the catheter assembly of FIG. 26 through line 26-26′.

FIG. 37 shows a side view of a distal portion of a catheter assembly.

### DETAILED DESCRIPTION

The following description and the accompanying drawings provide examples for the purposes of illustration. However, these examples should not be construed in a limiting sense as they are not intended to provide an exhaustive list of all possible implementations.

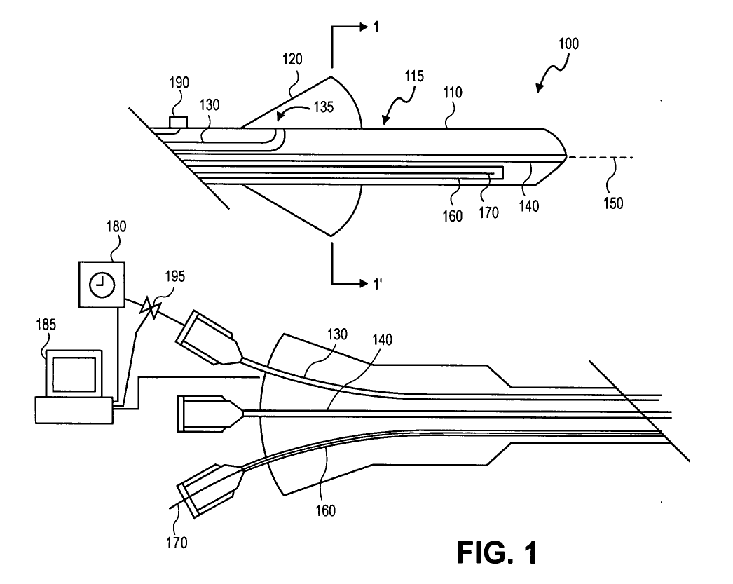

Referring now to FIG. 1, a side cross-sectional view of a portion of a catheter assembly suitable for insertion into a vessel (such as a blood vessel) of a subject is shown. Specifically, catheter assembly 100 includes primary cannula 110. Primary cannula 110 is of a size (e.g., outer diameter) and length suitable to be advanced through the vasculature of a human subject, such as through the femoral artery to a position within the cardiovascular system of a human subject.

Primary cannula 110 includes cannula 130 extending from a proximal end to a distal portion of catheter assembly 100. Cannula 130 has a lumen therethrough with lumen opening 135 on outer surface 115 of primary cannula 110. A proximal end of cannula 130 has a port to accommodate a solution into the lumen of cannula 130. Representatively, a flushing solution (e.g., injectate) may be introduced into a vessel via cannula 130.

Catheter assembly 100 illustrated in FIG. 1 also includes fluid dispersion device 120 connected to outer surface 115 of primary cannula 110. In one embodiment, fluid dispersion device 120 is generally arc-shaped and can, depending on the construction, disperse the injectate in a uniform or a non-uniform manner throughout a flow of fluid (e.g., blood) in which primary cannula 110 is disposed. Thus, fluid dispersion device 120 helps mix the injectate with blood flow in the vessel in order to avoid some of the problems discussed above when a streamline of injectate is introduced into a laminar flow of blood.

Fluid dispersion device 120, in one embodiment, is a conical structure with an apex directed proximally and a base directed distally. A diameter of the base of fluid dispersion device, in one embodiment, is large enough to disrupt the laminar flow patterns of blood in a blood vessel but not large enough to totally occlude the vessel. A representative diameter of a base of fluid dispersion device 120 is on the order of two millimeters (mm). It is appreciated that the diameter may vary depending at least in part on the diameter of a vessel where fluid dispersion device 120 is to be deployed. Representatively, fluid dispersion device 120 is a biocompatible polymer that may be collapsed within a removable sheath. Suitable materials for fluid dispersion device 120 include, but are not limited to, polyesters, polyethylene, nylon, polyether block amider (e.g., PEBAX®, commercially available from Elf Atochem of Avon, N.J.) or other catheter materials. Once catheter assembly is placed within a vessel at a region of interest, the sheath may be retracted or removed to expose fluid dispersion device 120. Fluid dispersion device 120 may then expand to a position such as shown in FIG. 1 where the base of fluid dispersion device has a diameter greater than an apex. This expansion may result from a material from which fluid dispersion device 120 is constructed (e.g., a shape memory material or frame of fluid dispersion device 120) or by a mechanical action on fluid dispersion device 120 (e.g., actuatable wire(s) connected to a base of fluid dispersion device and extending beyond a proximal end of catheter assembly 100). Alternatively, fluid dispersion device 120 may have an apex and base of similar diameter (perhaps the diameter of the base is slightly larger than a diameter of the more proximal apex). In this case, a sheath may not be required. Additionally, if the fluid dispersion device is made of a suitably elastic material, the base diameter may be designed to expand during flushing, under the pressure of the flush, and, after the flush, to return to its original (or close to its original) diameter close to an outer diameter of primary cannula 110.

As noted, in one embodiment, fluid dispersion device covers lumen opening 135. The dispensing of a flushing solution (injectate) through lumen opening 135 will cause the fluid to contact fluid dispersion device 120 and fluid dispersion device will direct the flushing solution around outer surface 115 of primary cannula 110. The dispensed flushing solution will travel distally beyond a base of fluid dispersion device 120 and disperse blood at least from the region distal to lumen opening 135.

In the embodiment shown in FIG. 1, catheter assembly 100 further includes an imaging/treatment device (e.g., a light-emitting device or an ultrasound device) capable of imaging (e.g., generating an image) or directing light at a blood vessel at a point or region distal to lumen opening 135 (e.g., to the right of lumen opening 135 as shown) so that the imaging/treatment device may image and/or treat at least a portion of the vessel in which primary cannula 110 is disposed. In this example, primary cannula 110 includes cannula 160 and imaging/treatment device 170 disposed in a lumen of cannula 160. Cannula 160 extends, in this embodiment, from a proximal end of catheter assembly 100 to at least a point distal to fluid dispersion device 120.

Primary cannula 110, in one embodiment, also includes guidewire cannula 140 extending from a proximal end to a distal end of primary cannula 110 in an over-the-wire (OTW) configuration. In an alternate embodiment, the guidewire may engage the catheter assembly 100 in a tip monorail distal to the travel of the imaging/treatment device 170 in cannula 160 in a manner similar to some IVUS catheter designs. In another embodiment the guidewire may engage the catheter assembly in a rapid exchange (RX) design similar to those of angioplasty catheters. In another embodiment, a catheter assembly may not include a separate imaging cannula, instead allowing a guidewire cannula to serve as an imaging or treating cannula (to accept an imaging or treatment device) once the catheter is placed at a region of interest and the guidewire removed.

The imaging/treatment capabilities of a device such as imaging/treatment device 170 (e.g., an OCT device or an IVUS device) disposed distal to the lumen opening can be improved due to the presence of flushing solution or injectate introduced into the imaging field/treatment area of the device via the lumen opening. As used herein, unless specifically described, references to an “imaging/treatment device” are intended to mean any one of the following: a single device capable of imaging and treating (e.g., photodynamic therapy), a device capable of imaging, and a device capable of treating.

FIG. 1 shows timer 180 that may be connected to a flushing solution or an injectate source and regulate introduction of a flushing solution or an injectate into cannula 130 from at least one lumen opening defined by the cannula into a flow of fluid in the vessel. Among other features, timer 180 may, for example, be connected to valve 195 and actuate the valve to regulate introduction of an injectate at a predetermined portion of a cardiac cycle of a subject. In this manner, injectate may be, for example, introduced into the flow of blood during a low flow rate portion of the natural pulsatile flow rate of blood within the subject. Introducing the injectate at a low flow rate portion of the cardiac cycle reduces the amount of injectate needed in order to effectively flush the vessel for imaging/treatment for at least a certain portion of time.

In addition, timer 180 may be used to regulate introduction of the injectate for a predetermined amount of time. For example, once the system determines that injectate should be introduced, the timer may be used to regulate how long the injectate is introduced into the blood flow (e.g., for a predetermined number of seconds and/or for a number of complete or partial cardiac cycles).

In the embodiment shown in FIG. 1, in addition to timer 180, a system includes processor 185, flow rate velocity sensor(s) 190 (e.g., disposed on primary cannula 110) and/or electrocardiogram (ECG) input. Timer 180, valve 195, flow rate velocity sensor(s) 190 and/or ECG input are connected to processor 185. Processor 185 includes machine readable instructions to control valve 195 based on inputs from timer 180, flow rate velocity sensor(s) 190 and/or ECG input. These components may be used to determine an appropriate time to introduce the injectate, taking into account flow velocity and distance between the lumen opening and the controlled and known beam path of an imaging/treatment device (e.g., the system contains an imaging/treatment device position system), such that the injectate will be located within the desired beam path at a desired time for imaging/treatment. Furthermore, the system may be used to regulate the duration of injectate introduction and to image/treat an entire portion of a vessel based on a composite of partial images/treatments, which may be obtained/performed at different times during one or more cardiac cycles. Additionally, information provided to processor 185 by velocity sensor(s) 190 (proximal to lumen opening 135) can be used to regulate a flush injection flow rate, such that machine-readable instructions of processor 185 may coordinate that the sensed flow rate is low, zero, or slightly negative (reverse flow) during the injection duration to ensure that the flush displaces a blood in the vessel using a minimum amount of injectate. In an imaging system, velocity sensor(s) 190 may not be required in some embodiments, as the delay of previous image clearances with the positions of lumen opening 135 and imaging device 170 provides sufficient information to estimate future delays when an injection of injectate is introduced at the same point in the ECG. Such systems may reduce the overall amount of flush needed by interposing periods of injectate flow with periods of blood flow and/or limiting the injectate flow rate to the minimum required to displace blood, which can reduce the risk of creating ischemic conditions in the subject. Certain cardiac irregularities may be sensed and taken into account in calculating the necessary delay times and the timing and duration of injectate introduction or to abort and/or repeat the affected injectate cycle(s).

FIG. 2 shows a cross-sectional view of catheter assembly 100 at line 1-1′ (looking distally). In this view, catheter assembly 100 includes primary cannula 110 with a lumen thereof including guidewire cannula 140 and imaging cannula 160. Catheter assembly 100 also includes fluid dispersion device 120 connected to primary cannula 110. In this embodiment, fluid dispersion device 120 has a generally arc shape with an outside diameter of its base less than an inside diameter of the vessel in which catheter assembly 100 is placed. As noted, the difference in diameters of a proximal end (apex) and distal end (base) of fluid dispersion device 120 may not be significant. In another embodiment, fluid dispersion device 120 need not completely surround primary cannula 110. Representatively, fluid dispersion device may be in the form of a flap covering lumen opening 135 and extending around and connected to less than an entire circumference of primary cannula 110. FIG. 2 shows blood vessel 200 (in ghost lines) in which catheter assembly 100 might be located. As shown, with fluid dispersion device 120 in an open or expanded position, blood vessel 200 is not completely occluded.

Besides fluid dispersion device 120 of catheter assembly 100 in FIG. 1 and FIG. 2, a catheter assembly (such as catheter assembly 100) may alternatively or additionally include other types of protrusions disposed on an outer surface of a catheter or primary cannula to improve dispersal/mixing of the flushing solution with the flow of blood in the vessel. In terms of catheter assemblies including multiple cannulas, the catheter or primary cannula is that cannula having an external surface that is in contact with fluid in a vessel (e.g., in contact with blood in an artery or vein). In one embodiment, a catheter or primary cannula has a lumen suitable to contain a portion of an imaging device (e.g., an OCT or other device) and an injectate cannula to introduce an injectate therethrough.

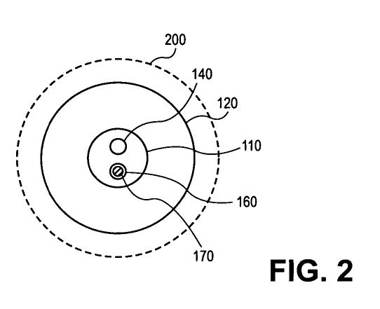

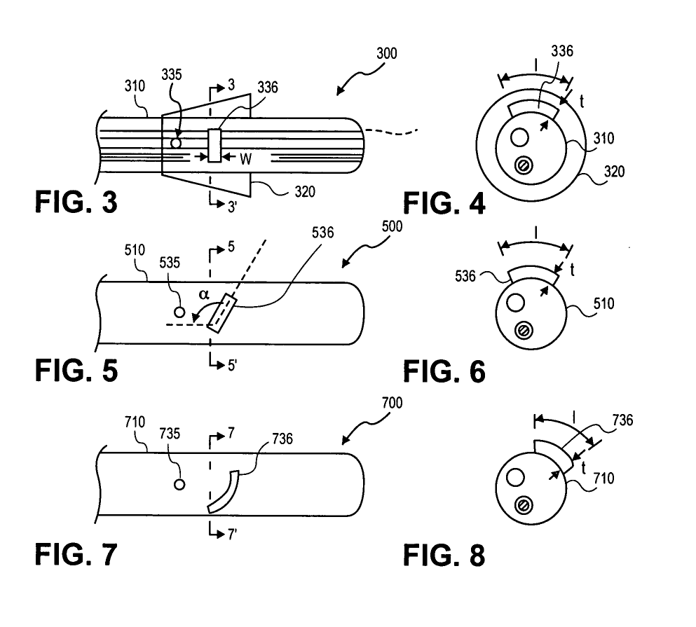

FIGS. 3-14 show various embodiments of different types of protrusions that may be used as fluid dispersion devices. For example, FIG. 3 shows a top view of catheter assembly 300 including primary cannula 310 including an injectate cannula terminating in lumen opening 335. An external surface of primary cannula 310 also has protrusion 336 raised at a right angle to the curved surface of primary cannula 310 (e.g., projecting out of the page towards the reader) at a point disposed distal to lumen opening 335. FIG. 4 shows a cross-section of the assembly of FIG. 3 through line 3-3′. In one embodiment, protrusion 336 has a rectangular shape with a length, l, that is generally orthogonal to a length of primary cannula 310. Protrusion 336 has a width, w, sufficient at least to provide structural integrity to protrusion 336 in the presence of blood flow in a vessel. Finally, protrusion 336 has a thickness, t, sufficient to disrupt laminar streamlines flowing in a distal direction relative to catheter assembly 300. A representative thickness is on the order of 0.5 mm. In the embodiment shown, fluid from lumen opening 335 would contact protrusion 336 at approximately a 90 degree angle, which may cause significant disruption of the laminar flow of injectate from lumen opening 335.

In the embodiment shown in FIG. 3, catheter assembly 300 also includes fluid dispersion device 320 connected at a proximal end to primary cannula 310. A proximal end of fluid dispersion device 320 is connected proximal to lumen opening 335. Lumen opening 335 is proximal to protrusion 336. A distal end of fluid dispersion device 320 covers a portion, including an entire portion (width and length portions) of protrusion 336. In this manner, fluid dispersion device 320 inhibits contact between protrusion 336 and a vessel wall. Individually and collectively, fluid dispersion device 320 and protrusion 336 may distribute a flush solution (e.g., injectate) from lumen opening 335 circumferentially around primary cannula 310.

FIG. 5 shows an alternative configuration of a catheter assembly. FIG. 6 shows a cross-section of the catheter assembly through line 5-5′. In this embodiment, catheter assembly 500 includes primary cannula 510 including an injectate cannula terminating in lumen opening 535. Primary cannula 510 also includes protrusion 536 having a length, l, width, w, and thickness, t, similar to the embodiment described with reference to FIG. 3. In this example, protrusion 336B is placed at a non-orthogonal angle relative to a length of primary cannula 510 (e.g., an angle a greater than 90 degrees). Catheter assembly 500 may also include a fluid dispersion device connected to primary cannula 510, for example, a configuration similar to the configuration described with reference to FIG. 3 and FIG. 4.

FIG. 7 shows another alternative configuration of a catheter assembly. FIG. 8 shows a cross-section of the catheter assembly, through line 7-7′ of FIG. 7. In this embodiment, catheter assembly 700 includes primary cannula 710 including an injectate cannula terminating in lumen opening 735. Primary cannula 710 also includes protrusion 736 on a surface of primary cannula 710 distal to lumen opening 735. In this example, protrusion 736 has a quarter quadrant arc shape. Protrusion 736 has a length, l, width, w, and thickness, t, sufficient to disrupt laminar streamlines. Catheter assembly 700 may also include a fluid dispersion device connected to primary cannula 710, for example, a configuration similar to the configuration described with reference to FIG. 3 and FIG. 4.

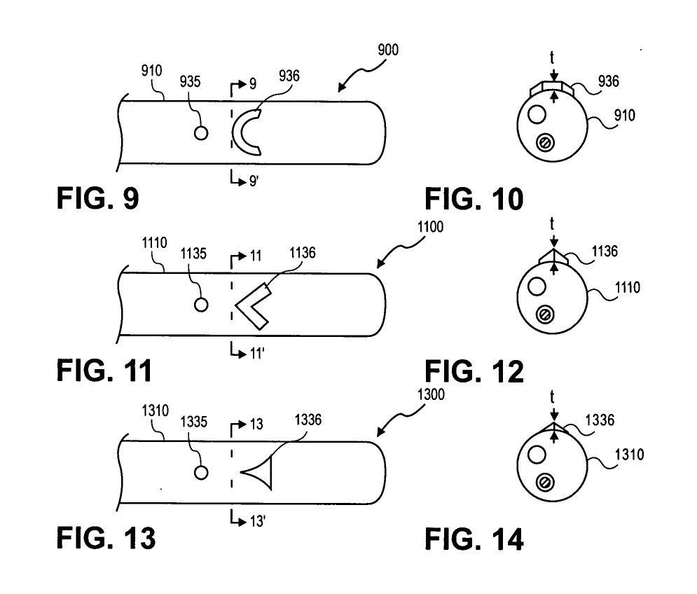

FIG. 9 shows another alternative configuration of a catheter assembly. FIG. 10 shows a cross-section of the catheter assembly through line 9-9′ of FIG. 9. In this embodiment, catheter assembly 900 includes primary cannula 910 including an injectate cannula terminating in lumen opening 935. Primary cannula 910 also includes protrusion 936 on a surface of primary cannula 910 distal to lumen opening 935. In this example, protrusion 936 has a half quadrant (e.g., semi-circle or arch) shape. Catheter assembly 900 may also include a fluid dispersion device connected to primary cannula 910, for example, a configuration similar to the configuration described with reference to FIG. 3 and FIG. 4.

FIG. 11 shows another alternative configuration of a catheter assembly. FIG. 12 shows a cross-section of the catheter assembly through line 11-11′ of FIG. 11. In this embodiment, catheter assembly 1100 includes primary cannula 1110 including an injectate cannula terminating in lumen opening 1135. Primary cannula 1110 also includes protrusion 1136 on a surface of primary cannula 1110 distal to lumen opening 1135. In this example, protrusion 1136 has an arrow head shape. Catheter assembly 1100 may also include a fluid dispersion device connected to primary cannula 1110, for example, a configuration similar to the configuration described with reference to FIG. 3 and FIG. 4.

FIG. 13 shows another alternative configuration of a catheter assembly. FIG. 14 shows a cross-section of the catheter assembly through line 13-13′ of FIG. 13. In this embodiment, catheter assembly 1300 includes primary cannula 1310 including an injectate cannula terminating in lumen opening 1335. Primary cannula 1310 also includes protrusion 1336 on a surface of primary cannula 1310 distal to lumen opening 1335. In this example, protrusion 1336 has a triangular shape. Catheter assembly 1300 may also include a fluid dispersion device connected to primary cannula 1310, for example, a configuration similar to the configuration described with reference to FIG. 3 and FIG. 4.

Different shapes, configurations, orientations, types, and numbers of fluid dispersion devices including one or more protrusions on an outer surface of a primary cannula may be used alone or in combination to disperse injectate into a flow of fluid within a vessel to improve the imaging/treatment capabilities of a device within a catheter. The various protrusions shown in FIGS. 3-14 are especially practical and effective when used inside a fluid dispersion device such as fluid dispersion device 320, as described with reference to FIG. 3 and FIG. 4.

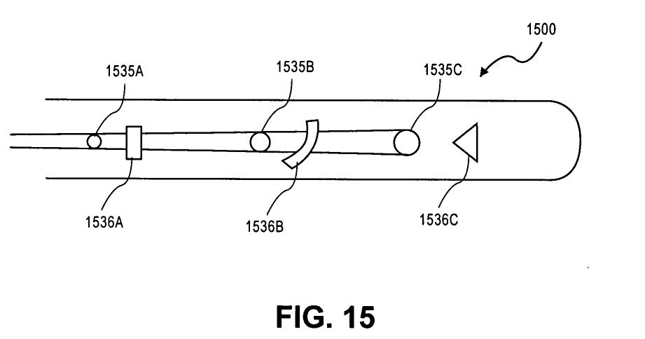

FIG. 15 shows another embodiment of a catheter assembly including a primarily cannula having a number of lumen openings suitable for dispensing a fluid (injectate) into a vessel and a number of protrusions. Catheter assembly 1500 includes primary cannula 1510 with three lumen openings disposed along at least a portion of the length of the cannula (e.g., a distal portion). Referring to FIG. 15, lumen opening 1535A is disposed most proximally along the portion of primary cannula 1510. Lumen opening 1535B is disposed distal from lumen opening 1535A and has a greater diameter than lumen opening 1535A. Finally, lumen opening 1535C is located distal to both lumen opening 1535A and lumen opening 1535C and is greater in diameter than both lumen opening 1535A and lumen opening 1535B. Primary cannula 1510 also includes, in this embodiment, multiple protrusions on a surface of primary cannula 1510, each protrusion distal to a respective lumen opening. FIG. 15 shows protrusion 1536A distal to lumen opening 1535A, protrusion 1536B distal to lumen opening 1535B and protrusion 1536C distal to lumen opening 1535C.

One advantage of the distally increasing size of each of multiple lumen openings is that a single lumen may provide injectate to be released through each of the lumen openings. FIG. 15 shows cannula 1530 feeding lumen opening 1535A, lumen opening 1535B and lumen opening 1535C. In such a configuration, it may be desirable to have lumen openings that are larger in a distal area in order to obtain a substantially uniform flow rate through each of the lumen opening since the pressure of injectate at each subsequent (e.g., more distal) opening will tend to decrease due to earlier pressure reductions from releasing injectate from the more proximal lumen openings. However, in other embodiments, each of the plurality of lumen openings may each have the same size, or the more distal lumen openings may have a smaller diameter than the more proximal lumen openings.

In another embodiment, a catheter assembly such as catheter assembly 1500, having multiple lumen openings on a cannula (such as primary cannula 1510), includes one or more fluid dispersion devices similar to fluid dispersion device 120 described with reference to FIG. 1 or fluid dispersion device 320 of FIG. 3. The one or more fluid dispersion devices, in one embodiment, would be disposed over one or more lumen openings and one or more protrusions.

In the embodiment shown in FIG. 15, each of the lumen openings appear approximately linearly aligned on a surface of primary cannula 1510. In another embodiment, a primary cannula having multiple lumen openings may not have the lumen openings linearly aligned on a surface of the primary cannula. Instead, the lumen openings may be at different circumferential positions along a cannula.

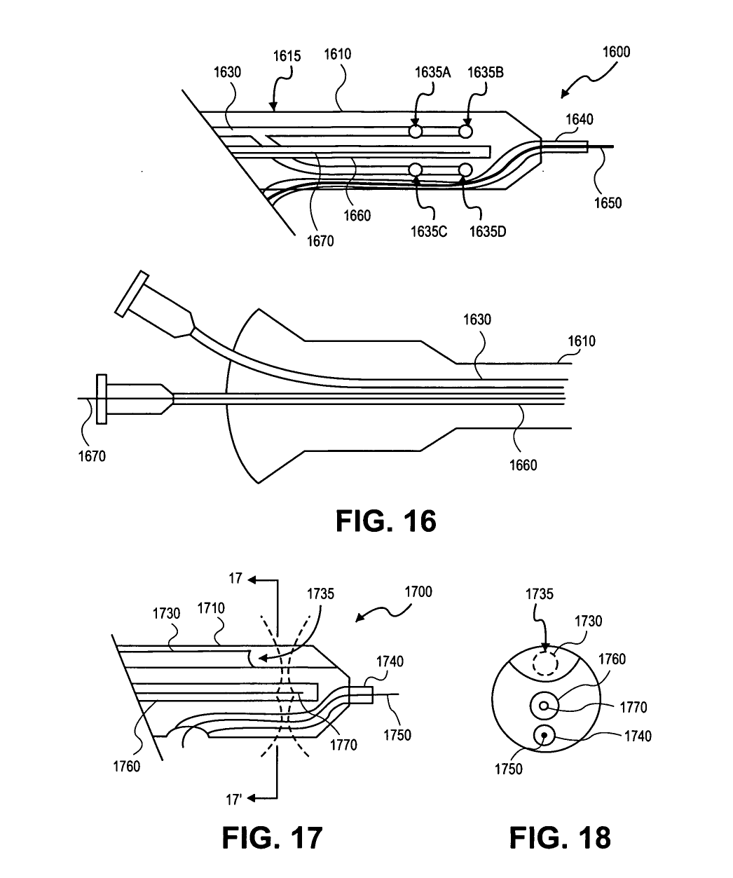

FIG. 16 shows a side cross-sectional view of a portion of a catheter assembly suitable for insertion into a blood vessel (such as a blood vessel of a subject). Catheter assembly 1600 includes primary cannula 1610. Primary cannula 1610 is of a size (e.g., outer diameter) suitable to be advanced through the vasculature of a human subject and positioned at a region of interest within the vasculature. Primary cannula 1610 includes cannula 1630 extending from a proximal end to a distal portion of the catheter assembly 1600. Cannula 1630 has a lumen therethrough with multiple lumen openings 1635A, 1635B, 1635C, and 1635D on outer surface 1615 of primary cannula 1610. A proximal end of cannula 1630 has a port to accommodate a flushing solution (e.g., injectate) into the lumen of cannula 1630. As illustrated, lumen openings 1635A, 1635B, 1635C, and 1635D are at different circumferential as well as longitudinal positions along primary cannula 1610. Representatively, lumen opening 1635A and lumen opening 1635C are at a similar longitudinal position and lumen opening 1635B and lumen opening 1635D are at a similar longitudinal position. A circumferential position of lumen opening 1635A and lumen opening 1635B is different than a circumferential position of lumen opening 1635C and lumen opening 1635D. It is noted that the circumferential position of lumen opening 1635A and lumen opening 1635B (or lumen opening 1635C and lumen opening 1635D) need not be the same. In the embodiment illustrated, cannula opening 1630 feeds all lumen openings. In the embodiment illustrated, cannula 1630 forks into two cannula portions at a distal portion of catheter assembly 1600.

Catheter assembly 1600 is a rapid exchange (RX) type catheter. In this manner, catheter assembly 1600 includes guidewire cannula 1640 at a distal portion of the catheter assembly. Guidewire 1650 enters a distal portion of primary cannula 1610 into a lumen of cannula 1640 within primary cannula 1610 and exits through cannula 1640 at a distal end. Catheter assembly also includes cannula 1660 disposed within primary cannula 1610. Cannula 1660 extends, in this embodiment, from a proximal end of catheter assembly 1600 to at least a point distal to lumen openings 1635A, 1635B, 1635C, and 1635D. Imaging/treatment device 1670 is disposed in a lumen of cannula 1660.

FIG. 17 shows another embodiment of a catheter assembly illustrating a distal portion of the catheter assembly. FIG. 18 shows a cross-sectional view through line 17-17′ of FIG. 17. Catheter assembly 1700 includes primary cannula 1710 having a lumen therethrough. Disposed within a lumen of primary cannula 1710 is cannula 1730, cannula 1740 and cannula 1760. Cannula 1730 extends to a proximal end of primary cannula 1710 and includes a port at a proximal end to accommodate a flushing solution (e.g., injectate) into a lumen of cannula 1730. Cannula 1740 extends from an opening in a distal portion of primary cannula 1710 and has a lumen suitable to accommodate a guidewire in a rapid exchange (RX) type catheter assembly. FIG. 17 shows guidewire 1750 within cannula 1740. Cannula 1760 extends from a proximal end to a distal portion of primary cannula 1710 and has a lumen to accommodate imaging/treatment device 1770 (e.g., OCT, IVUS).

Referring to FIG. 17 and FIG. 18, in this embodiment, cannula 1730 distally terminates at a point proximal to the distal end of primary cannula 1710. The distal termination of cannula 1730 provides lumen opening 1735. Primary cannula 1710 is cutaway at lumen opening 1735. In this configuration, cannula 1730 and lumen opening 1735 provide a distal forward flush configuration (i.e., a flushing solution (e.g., injectate) is introduced in a distal rather than lateral or radial direction). In one embodiment, a beam path of imaging/treatment device 1770 is located distal to lumen opening 1735. In this manner, a solution (e.g., injectate) is introduced proximal to the beam path.

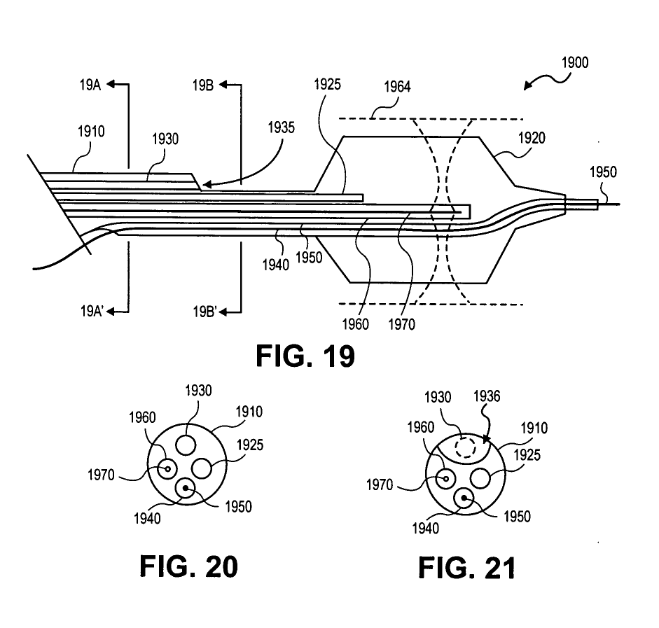

FIG. 19 shows a distal portion of another embodiment of a catheter assembly. Catheter assembly 1900 includes primary cannula 1910 and inflatable balloon 1920 connected to a distal end of primary cannula 1910. Primary cannula 1910 has a lumen therethrough that accommodates cannula 1930, inflation cannula 1925, cannula 1940, and cannula 1960. FIG. 20 shows a cross-sectional view of catheter assembly 1900 through line 19A-19A′. FIG. 21 shows a cross-sectional side view through line 19B-19B′ of FIG. 19.

In the embodiment shown in FIGS. 19-21, cannula 1930 extends from a proximal end to a distal portion of catheter assembly 1900. Cannula 1930 has a lumen therethrough with lumen opening 1935 from primary cannula 1910 directed in a distal direction at a point proximal to balloon 1920. Primary cannula is cut away at lumen opening 1935. Representatively, a flushing solution (e.g., injectate) may be introduced into a vessel in a forward flush configuration via cannula 1930.

In the embodiment of catheter assembly 1900 shown in FIGS. 19-20, balloon 1920 is in an inflated state. Representatively, cannula 1925 is a balloon inflation cannula and has a lumen therethrough to introduce an inflation fluid to inflate balloon 1920. In this embodiment, balloon 1920 is inflated or expanded to partially occlude a flow of fluid within vessel 1964.

Cannula 1960 has a lumen therethrough to accommodate imaging/treatment device 1970. Cannula 1960 extends, in one embodiment, from a proximal end of catheter assembly 1900 to a position within balloon 1920. In this manner, imaging/treatment device 1970 has a beam path through balloon 1920 and distal to lumen opening 1935 where a flush solution is introduced into vessel 1964. With this configuration, a flushing solution would tend to remove blood flow around balloon 1920 and thus the flush volume required for imaging/treatment may be reduced. For imaging/treating long vessel segments, catheter assembly 1900 may be placed at a distal end of a desired visualization/treatment portion of vessel 1964 and pulled proximally. For technologies like infrared spectroscopy or intravascular magnetic resonance imaging (MRI) that do not require a blood free field, catheter assembly 1900 may be used without a flushing solution (e.g., without cannula 1930). In this situation, catheter assembly 1900 would be suitable to center the imaging device within the blood vessel. Further, to reduce the profile of catheter assembly 1900, in another embodiment, inflation cannula 1925 may be combined with cannula 1960 or cannula 1940 provided proper seals are utilized at a proximal end of the catheter assembly.

As noted above, primary cannula 1910 also includes cannula 1940. Cannula 1940 extends from a distal portion to a distal end of catheter assembly 1900 and has a lumen therethrough to accommodate guidewire 1950 in a rapid exchange (RX) configuration.

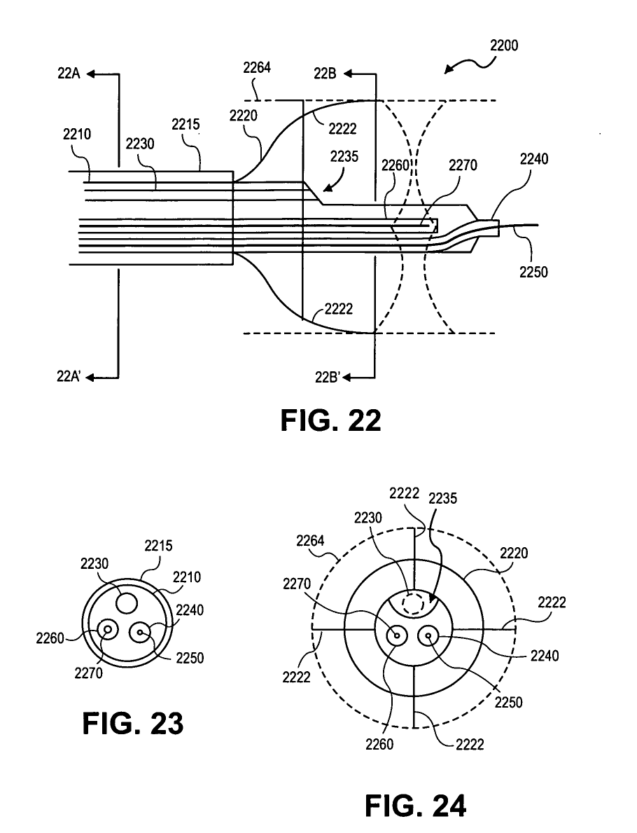

FIG. 22 shows another embodiment of a catheter assembly. Catheter assembly 2200 includes primary cannula 2210 having a lumen therethrough. Primary cannula 2210 includes cannula 2230 extending from a proximal end to a distal portion of catheter assembly 2200. Cannula 2230 has a lumen therethrough with lumen opening 2235 directed distally in a flush forward configuration. Representatively, a flushing solution (e.g., injectate) may be introduced into a vessel via cannula 2230.

Primary cannula 2210 of catheter assembly 2200 also includes cannula 2240 having a lumen therethrough to accommodate guidewire 2250. In the embodiment illustrated, catheter assembly 2200 is an over-the-wire (OTW) configuration with cannula 2240 extending from a proximal end to a distal end of primary cannula 2210. Primary cannula 2210 also includes cannula 2260 having a lumen therethrough to accommodate imaging device 2270. In one embodiment, cannula 2260 extends from a proximal end of primary cannula 2210 to a distal portion of primary cannula 2210.

Catheter assembly 2200 illustrated in FIG. 22, also includes fluid dispersion device 2220. In one embodiment, fluid dispersion device 2220 includes framework or scaffold 2222 covered by a non-porous material (e.g., a non-porous polymer material). Framework 2222 can resemble flower petals, a basket, or a cage. Framework 2222 may be made of a shape memory material such as a nickel-titanium alloy (e.g., nitinol) ribbon or wire. Representatively, framework 2222 may be three or more ribbons sized relative to a vessel diameter. Catheter assembly 2200 includes sheath 2215 over primary cannula 2210. In one embodiment, sheath 2215 extends over fluid dispersion devise 2220 (including any extending framework 2222) and confines fluid dispersion device to a diameter consistent with an inner diameter of sheath 2215. Sheath 2215 may be retracted to expose fluid dispersion device 2220. In the embodiment where framework 2222 is a shape memory material, the exposure of fluid dispersion device 2220 within vessel 2264 will cause fluid dispersion device 2220 to expand to a shape memory position.

FIG. 22 shows catheter assembly 2200 with fluid dispersion device 2220 exposed from sheath 2215 and in an expanded position. If a diameter of framework 2222 is greater than an inner diameter of vessel 2264 at a deployment site, then primary cannula 2210 will be forced into the center of the vessel lumen. If a diameter of framework 2222 of fluid dispersion device 2220 is less than a diameter of vessel 2264 at a deployment site, only a portion of fluid dispersion device 2220 will contact a vessel wall and minimize the shifting of primary cannula 2210.

FIG. 23 shows a cross-sectional side view through line 22A-22A′ of FIG. 22. FIG. 23 shows sheath 2215 surrounding primary cannula 2210 at a location proximal to fluid dispersion device 2220. FIG. 24 shows a cross-sectional side view through line 22B-22B′ of FIG. 22 at a point distal to fluid dispersion device 2220. FIG. 24 shows framework 2222 (four ribbons) of fluid dispersion device 2220 contacting vessel 2264. FIG. 24 also illustrates a gap or space between the body of fluid dispersion 2220 and blood vessel 2264. In this manner, only framework 2222 contacts blood vessel 2264 and the gap between a body of fluid dispersion device 2220 and blood vessel 2264 allows blood flow through the vessel. It is appreciated that fluid dispersion device 2220 may be sized for a particular blood vessel. Alternatively, sheath 2215 (e.g., the retraction of sheath 2215) may be utilized to control the expanded diameter of fluid dispersion device 2220 within vessels of different sizes.

Referring again to FIG. 22, in the embodiment illustrated, lumen opening 2235 for a flushing solution (e.g., injectate) is disposed distal to fluid dispersion device 2220. Similarly, a beam path of imaging/treatment device 2270 is disposed distal to fluid dispersion device 2220. A beam path of imaging/treatment device 2270 is also disposed distal to lumen opening 2235. In this manner, fluid dispersion device 2220 may reduce the blood flow past an imaging/treatment site and a flushing solution (e.g., injectate) may be used to remove blood from an imaging/treatment site to improve the imaging/treatment capabilities of the catheter assembly. For imaging/treatment of extended or long blood vessel sections, catheter assembly 2200 may be deployed at a distal position and advanced proximally (e.g., pulled) with fluid dispersion device 2220 deployed and a flushing solution injected from lumen opening 2235.

Catheter assembly 2200 may have a number of variations. One variation includes introducing a flushing solution through sheath 2215 (i.e., through a lumen of sheath 2215 defined by a space between primary cannula 2210) and an inner diameter of sheath 2215. In another variation, a body of fluid dispersion device 2220 may be made of a porous material (e.g., a porous polymer) to allow a flushing solution through sheath 2215 to flow through fluid dispersion device 2220. The pores of a porous material may be sized to regulate the blood flow and flush solution or potentially to allow flush solution to pass, but not blood (or to allow blood to pass at a much slower rate). Finally, catheter assembly 2200 may be utilized in embodiments where a flush is not required such as infrared spectroscopy or intravascular MRI. In such case, a fluid dispersion device may not require body 2220. In this case, fluid dispersion device 2220 may act as a centering device and require only framework 2222.

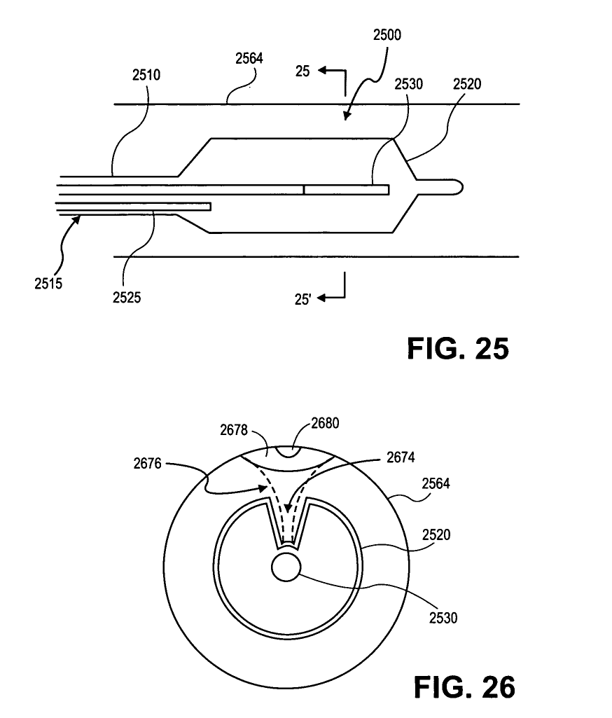

FIG. 25 shows a cross-sectional view of a distal portion of a catheter assembly. Catheter assembly 2500 includes catheter 2510 disposed within vessel 2564 of a subject. Catheter 2510 includes balloon 2520 connected thereto in an axial arrangement. FIG. 25 shows balloon 2520 inflated or expanded to partially occlude a flow of fluid within vessel 2564. The partial occlusion allows enough blood flow for an extended imaging (or treatment) time. The partial occlusion also provides balloon 2520 with an outer diameter (OD) that in an expanded configuration or state is away from the vessel wall, but close enough that the vessel wall can be imaged deep enough to visualize (or treat) a vulnerable plaque or other desired wall structure).

Catheter 2510 defines lumen 2515 through which inflation cannula 2525 may be positioned to deliver a fluid to inflate balloon 2520. Lumen 2515 of catheter 2510 also accommodates imaging/treatment device 2530 may be positioned along the length of catheter 2510 in order to image at least a portion of vessel 2564. As shown, balloon 2520 has already been inflated in order to partially occlude vessel 2564.

In one embodiment of catheter assembly 2500, balloon 2520 has a continuous outer diameter of similar dimension. FIG. 26 shows a second embodiment of catheter assembly 2500 in a cross-sectional view taken along line 25-25′ of FIG. 25. Balloon 2520 in this embodiment has channel 2674, which is substantially parallel to a longitudinal axis of catheter 2510 and extends along a medial or working length section of balloon 2520. Although only a single channel is shown, there may be two or more. This configuration allows blood flowing in vessel 2564 to pass through channel 2574 (e.g., selectively partially occluding vessel 2564). Thus, even if balloon 2520 fills vessel 2564 to the point that flow is minimal or non-existent in other areas around the balloon (a possible embodiment), the narrow channel(s) (e.g., channel 2674) allows enough blood flow for an extended imaging (or treatment) time. Balloon 2520 having one or more channels may be formed by balloon blowing techniques such as blowing a tubing into a mold of similar shape in a heated condition.

In one embodiment, the dimensions of channel 2674 in balloon 2520 are selected to permit blood flow through the channel without completely degrading the ability of imaging/treatment device 2530 to image/treat at least a portion of vessel 2564 aligned with the channel. Specifically, imaging/treatment device 2530 may have beam path 2676, that contains at least half the light energy of a phototherapy light beam, that is wider than channel 2674. Therefore, imaging/treatment device 2530 may be able to “see” and/or access significant characteristics of a wall of vessel 2564 despite a possible blind spot created by the blood flowing through channel 2674.

In one embodiment, imaging device 2530 can rotate about the center of catheter 2510. Alternatively, catheter 2510 on which balloon 2520 is mounted may be rotated to image (or treat) the previously blocked areas of the vessel wall. Thus, imaging/treatment device 2530 has the potential to form a 360 degree image of vessel 2564 (e.g., 360 degrees of the vessel circumference). Referring to FIG. 26, as illustrated, blood vessel 2564 has vulnerable plaque 2678 with lipid core 2680. Vulnerable plaque 2678 is directly aligned with channel 2674 of balloon 2520. A portion of vulnerable plaque 2678 may be blocked from view by blood flowing through channel 2674 (a blind spot). Having the capability to image/treat up to 360 degrees of the vessel circumference will allow a portion of vulnerable plaque 2678 to be detected even in this configuration.

In an embodiment shown in FIG. 25 and FIG. 26, balloon 2520 in an inflated or expanded state only partially occludes vessel 2564 (without channel 2674). One desirable feature of such construction is that the expanded balloon may not contact a vessel wall and thus the potential for vessel wall damage is reduced. The expanded balloon also reduces the path thickness of blood through the vessel. In other words, a continuous flow of blood past balloon 2520 will occupy a cross-sectional area determined by the inner diameter of vessel 2564 minus an outer diameter of balloon 2520. In one embodiment, a suitable cross-sectional area is defined by a radius on the order of one millimeter or less. A typical OCT imaging device will image about two millimeter (mm) or less into tissue or blood. With one millimeter of blood in a light path in a blood vessel, an OCT imaging device should be able to detect a vulnerable plaque or a plaque in danger of becoming a vulnerable plaque even if the true imaging depth capability of, for example, an OCT device is on the order of 1.2 mm to 1.7 mm.

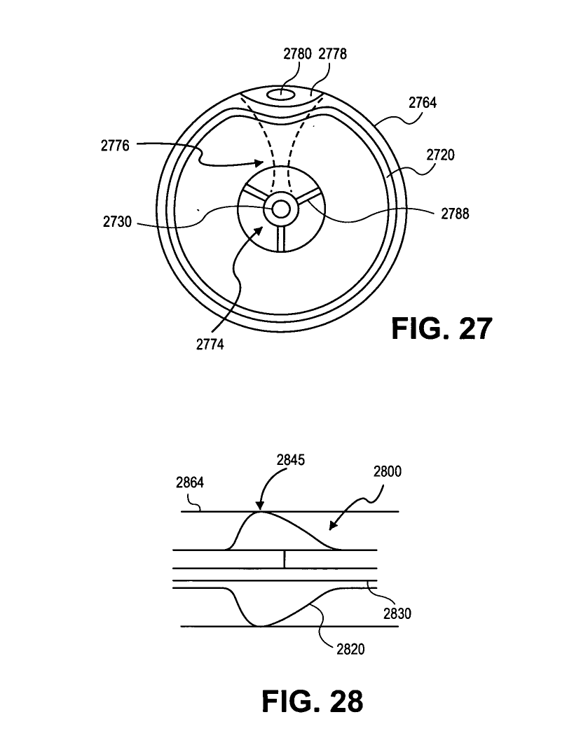

FIG. 27 shows an alternative cross-sectional embodiment of a catheter balloon to that shown in FIG. 26. Specifically, balloon 2720 is illustrated disposed within vessel 2764. The catheter assembly includes imaging/treatment device 2730 with an imaging/treatment portion (e.g., capable of generating beam path within balloon 2720). Channel 2774 is created by a gap between imaging/treatment device 2730 and balloon 2720. In one embodiment, the gap is maintained by supports 2788.

Imaging/treatment device 2730 has beam path 2776 capable, in one embodiment, as an imaging device of detecting vulnerable plaque 2778, including lipid core 2780, and/or other features of vessel 2764. Channel 2724 is designed so that the depth of blood through which imaging/treating device 2730 must image is small enough so as not to degrade the image obtained by imaging/treatment device 2730 and/or render the treatment from imaging/treatment device 2730 ineffective, taking into account the refractory effects of the blood on the light emitted by imaging/treatment device 2730 (e.g., an OCT or IVUS device). If imaging device 2730 is an OCT device, one target depth of blood through which an acceptable image may be obtained is about one millimeter.

In the embodiment illustrated in FIG. 27, an exterior surface of balloon 2720 in an expanded state contacts or may contact blood vessel 2764. To reduce the possibility of injury to a wall of a blood vessel, the balloon may be made compliant to achieve an expanded state at relatively low pressures compared to traditional angioplasty balloon materials and expansion pressures. Suitable materials for compliant balloons are described in commonly-owned, co-pending U.S. patent application Ser. No. 10/800,323, titled “Infusion Treatment Agents, Catheters, Filter Devices, and Occlusion Devices and Uses Thereof,” filed Mar. 11, 2004 which is incorporated herein by reference. Another option to reducing the possibility of injury to a wall of a blood vessel is to minimize the portion of the working length of the balloon that comes in contact with a vessel wall. One way this may be done is by tapering a diameter of the balloon. FIG. 28 shows catheter assembly 2800 having balloon 2820 and imaging/treatment device 2830. Catheter assembly 2800 may be similar to that described above with respect to FIG. 27 with a channel for blood flow defined between balloon 2820 and imaging/treatment device 2830. In this embodiment, in an expanded state only a proximal portion of a medial working length of balloon 2820 of contacts blood vessel 2864 (at point 2845). Distal to point 2845 (e.g., downstream in terms of blood flow), balloon 2820 tapers to a smaller diameter.

In the embodiments described with reference to FIGS. 25-28, the balloons may be of various lengths and embodiments include multiple balloons connected in series along a catheter. Increasing the length of a balloon or multiple balloons allows imaging of longer vessel lengths (e.g., vessel lengths on the order of five centimeters (cm)).

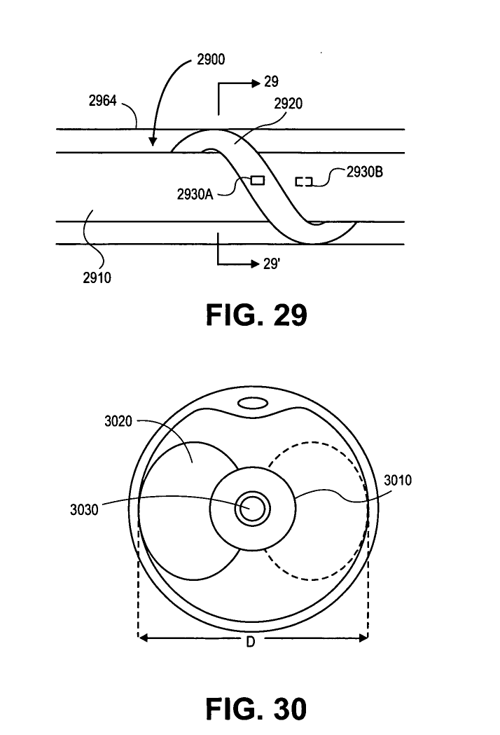

FIG. 29 shows an embodiment of a catheter assembly having a distal portion disposed in a blood vessel. Catheter assembly 2900 includes catheter 2910 disposed within vessel 2964 and spiral-shaped balloon 2920 that is wound around at least a portion of catheter 2910. Although only one section of spiral-shaped balloon is shown, balloon 2920 may have multiple inflated sections wrapped around catheter 2910 to guide and/or redirect the flow of blood through vessel 2964 (e.g., an alternative device to selectively partially occlude a vessel). Balloon 2920 may be connected to catheter 2910 by an adhesive or thermal fusion bonding.

FIG. 30 shows a cross-section of the catheter assembly of FIG. 29 through line 29-29′. In this embodiment, balloon 2920 may contact a portion of vessel 2964. The spiral configuration of balloon 2920 does not occlude vessel 2964 and blood may pass, in spiral paths, around balloon 2920.

FIG. 29 shows the placement of an imaging/treatment device. In one embodiment, imaging/treatment device 2930A such as photodynamic light source (e.g., OCT) is placed beneath the visible section of spiral-shaped balloon 2920 to image/treat at least a portion of vessel 2964 through spiral-shaped balloon 2920. In another embodiment, an imaging/treatment device (illustrated as imaging/treatment device 22930B) is placed distal to the visible section of spiral-shaped balloon 2920 in order to provide a light beam to an area of vessel 2964 distal to the visible section of spiral-shaped balloon 2920. Placement of an imaging device in either of the positions indicated by imaging/treatment device 2930A and imaging/treatment device 2930B may improve the ability of the imaging/treatment device to image/treat. Since the blood flow in vessel 2964 has been redirected to flow around balloon 2920, blood flow is allowed to continue in vessel 2964 increasing imaging/treatment time. Balloon 2920 tends to center imaging/treatment device 2930A or 2930B in the vessel, thus the maximum light path distance through the blood to a wall of vessel 2964 is limited to an acceptable distance (e.g., one millimeter). In the position indicated by imaging/treatment device 2930A, this distance is even shorter for a light path through a portion of balloon 2920 due to the presence of a fluid filled balloon (e.g., a balloon filled with an optically translucent fluid).

Any of the embodiments described with reference to FIGS. 1-30 and the accompanying text may be used to image a portion of a blood vessel by providing a light beam from an imaging device. To enhance the quality of the image provided by an imaging device or treatment provided by a treatment device, an injectate may be introduced, preferably proximal (in terms of blood flow) to the imaging device. Thus, the catheter designs shown with reference to FIGS. 1-30 may each include an injectate cannula terminating with a lumen opening, for example, proximal to or at a proximal portion of the occluding device (e.g., proximal to or at a proximal portion of a balloon). Examples of suitable injectate cannulas are described with reference to FIG. 1 and FIGS. 16-24 and the accompanying text.

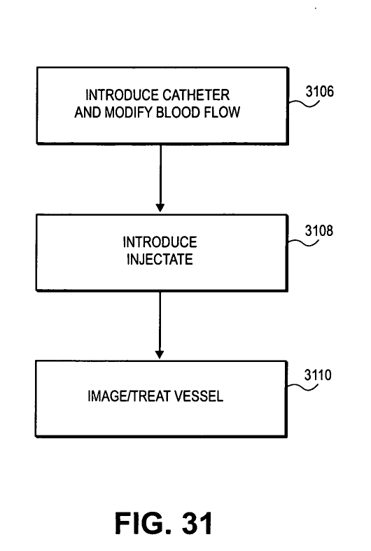

FIG. 31 shows a flow chart according to one embodiment of flushing a vessel. At block 3106, a catheter is introduced into a vessel of a subject, the catheter including a structure to modify a flow of fluid within the vessel. The manner in which the catheter modifies the flow of fluid within the vessel may include, for example, any of the devices and/or methods disclosed herein.

At block 3108, the introduction of an injectate from at least one lumen opening defined by the catheter is timed for proper introduction into a flow of fluid in the vessel. As described above, timing may include introducing the injectate at a predetermined/calculated portion of a cardiac cycle of the subject and/or introducing the injectate for a predetermined/calculated amount of time. Additionally, the rate of injectate flow may be predetermined/calculated/ adjusted as per sensor input.

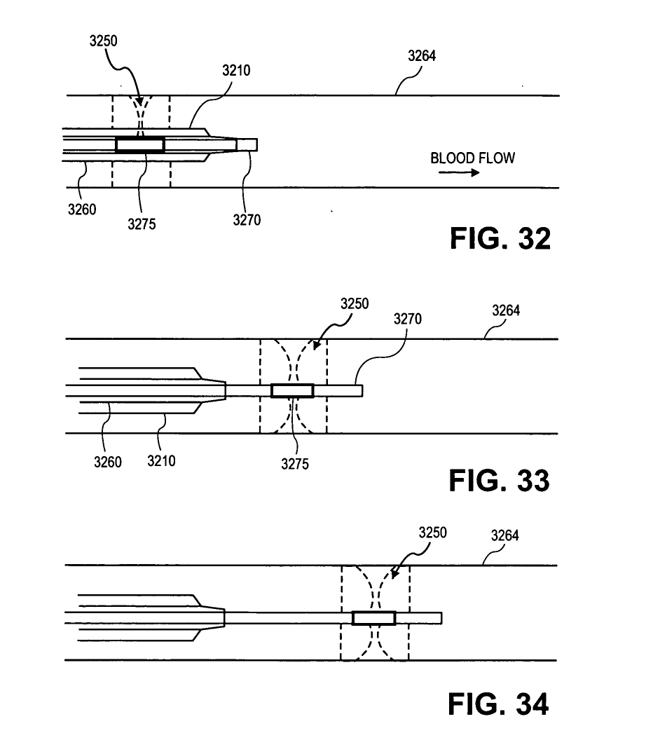

The method of FIG. 32 may additionally include, at block 3110, imaging/treating at least a portion of the vessel with an imaging/treatment device. If imaging/treating is included in the method, timing may include introducing the injectate such that the injectate is disposed within the imaging field/treatment area of the imaging/treatment device during a predetermined/calculated portion of a cardiac cycle of the subject. As described above, the flow rate of the blood within the vessel and the distance between the lumen opening and the light beam path may both be used to calculate the optimal time to introduce the injectate to maximize the amount of time during which the injectate is within the light beam path (e.g., time injectate introduction so injectate is within light beam path during low flow rate portion of cardiac cycle). By maximizing the time in which the injectate is in the light beam path, the overall procedure time may be reduced, and the amount of flush required for the procedure may be minimized.

FIG. 32 shows catheter or primary cannula 3210 disposed within vessel 3210 of a subject. Catheter 3210 includes cannula 3260 disposed in a lumen of catheter 3210. Imaging device 3270 is disposed in a lumen of cannula 3270. Imaging/treatment device 3270 is, for example, an OCT device including a fiber optic cable, refractive index gradient (GRIN) lens and prism/mirror. Imaging/treatment device 3270 includes imaging portion 3275. Imaging/treatment device 3270 is movable within imaging cannula 3260.

In one embodiment, an injectate may be introduced into vessel 3264 at a point proximal to imaging/treatment portion 3275 (to the left as viewed) of imaging/treatment device 3270. One suitable technique for introducing an injectate into vessel 3264 is through a cannula having a dispensing port in catheter 3210 proximal to imaging/treatment portion 3275 of imaging/treatment device 3270. The catheter assembly of FIG. 32 may also include one or more fluid dispersion devices and/or one or more balloons proximal to imaging/treatment portion 3275 of imaging/treatment device 3270. In this context, reference is made to FIGS. 1-30 and the accompanying text (with the possible exception of the fluid dispersion device described with reference to FIGS. 22-24 with extending framework).

In one embodiment, an injectate introduced (perhaps through the timing techniques discussed above) into vessel 3264 creates flush zone or bolus 3250 that moves in a distal direction within the blood vessel. As the bolus travels over imaging/treatment portion 3275 of imaging/treatment device 3270, the wall of blood vessel 3264 is imaged.

FIG. 33 shows the blood vessel of FIG. 32 at a later point in time. In this view, bolus 3250 has moved distally beyond catheter 3210. In one embodiment, imaging/treatment device 3270 may be advanced distally with bolus 3250 to provide push forward imaging. FIG. 33 shows imaging/treatment portion 3275 imaging/treating a portion of vessel 3264 that is distal to the portion imaged in FIG. 32. FIG. 34 shows vessel 3264 at a still later point in time and imaging portion 3275 at a point distal to a point shown in FIG. 32 and distal to a point shown in FIG. 33. The rate at which the bolus will travel may be predicted by a velocity sensor or ECG monitoring as described above.

With push forward imaging, the longitudinal motion of the imaging/treatment position (imaging/treatment portion 3275) follows bolus down the vessel. Using this technique may limit the number of boluses required to image a given length of vessel. In one embodiment, using a rate controlled push forward, a flush bolus of sufficient length and an OCT system with a sufficient scan rate, a single flush may be required to image/treat a desired vessel segment before the bolus reaches the arterioles/capillaries (which, as previously discussed, would necessitate a larger flush flow rate).

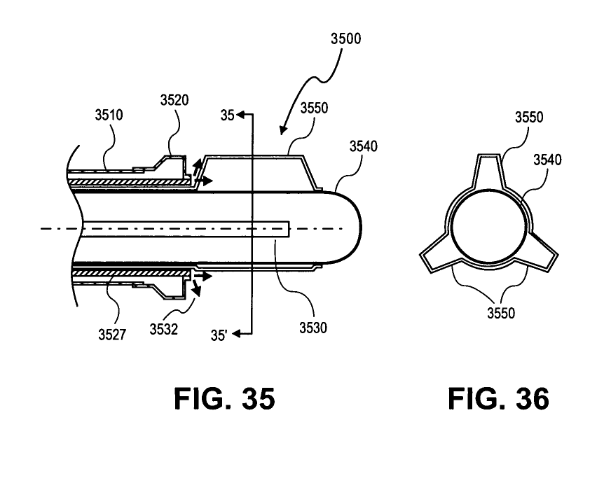

FIG. 35 shows a cross-sectional side view of a catheter assembly. Catheter assembly 3500 includes primary cannula 3510. Connected at a distal portion of primary cannula 3510 is balloon 3520. Disposed within a lumen of primary cannula 3510 and axially extending beyond balloon 3520 is centering catheter 3540. A distal end of centering catheter 3540 includes multi-lobed balloon 3550. FIG. 36 shows a cross-sectional view of centering catheter 3540 taken along line 35-35′.

As configured, imaging device 3530 is placed through a lumen of centering catheter 3540 and has an imaging/treatment portion that may direct a photodynamic light beam beyond a distal end of balloon 3520. In this manner, balloon 3520 may be used to modify/redirect/minimize blood flow proximal to a light beam path.

FIG. 35 shows catheter assembly 3500 lumen 3527 to receive centering catheter 3540. Lumen 3527 has a size (diameter) large enough that, in the presence of centering catheter 3540, may also be used to introduce flushing solution 3532 (e.g., saline solution or a blood substitute) into a vessel in which catheter is disposed.

Centering catheter 3540 includes multi-lobed balloon 3550. As shown, balloon 3550 is a tri-lobed balloon. However, other numbers, shapes, types, and configurations of balloons may be used in conjunction with centering catheter 3540.

In various embodiments, the lobes of balloon 3550 may have a fixed diameter or may be inflatable to align balloon 3550 within the vessel. Moreover, the lobes may be designed to minimize interference with imaging and/or photodynamic therapy applications (e.g., small separation between lobes).

In some applications (e.g., imaging and/or photodynamic therapy), it can be advantageous to align the imaging or therapy device with the longitudinal axis of the vessel. Centering catheter 3540 can assist in achieving this alignment.

For example, in some imaging applications (e.g., OCT) the imaging device may have a limit on how much blood can be present between the imaging device and the vessel wall before the image obtained by the imaging device is not satisfactory. For an OCT device, this depth is approximately one millimeter. In order to ensure the amount of blood between the imaging device and the vessel wall does not exceed this depth, centering catheter 3540 may be used to ensure that the imaging device, which may be located within centering catheter 3540, is substantially centered in a vessel within which catheter assembly 3500 is disposed.

For certain photodynamic therapy devices, it is often desirable that the therapy device is located at approximately the same distance from the areas being treated within the vessel. Thus, in many intravascular procedures, the therapy device can be substantially aligned along the longitudinal axis of the vessel in which the therapy device is disposed. The centering catheter shown in FIG. 35 and FIG. 36 can help to achieve this alignment.

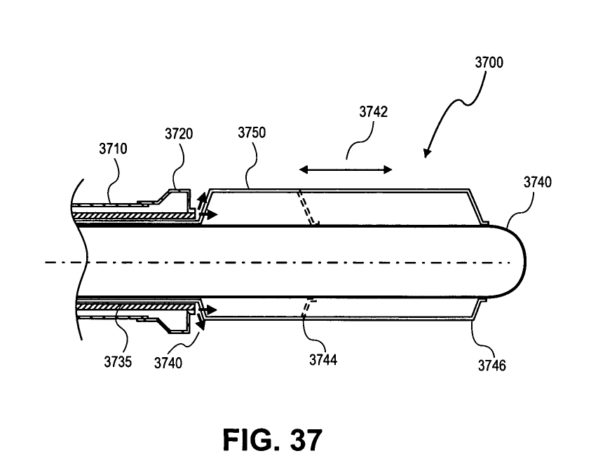

FIG. 37 shows another embodiment of a catheter assembly. Catheter assembly 3700 includes primary cannula 3710 having balloon 3720 connected to a distal end thereof. Primary cannula 3710 also includes lumen 3735 to receive centering catheter 3740. Centering catheter 3740 includes balloon 3750, which has a variable length.

The length of balloon 3750 may be varied by expanding or retracting in a distal or proximal direction, indicated by arrow 3742. The fully retracted position for balloon 3750 is indicated by position 3744. The fully expanded position for balloon 3750 is indicated by position 3746. In one embodiment, balloon 3750 may have a length between approximately 0.5 centimeters (“cm”) and 15 cm. However, lengths outside of this range could be used.

In various embodiments described above, a flushing solution or injectate is described in conjunction with imaging of a blood vessel. In one embodiment, a suitable injectate is water or a saline solution. In an alternative embodiment, a blood compatible, electromagnetic wave-transparent oxygen carrier (e.g., a blood substitute) may be introduced from the catheter into the vessel before and/or during imaging/treatment. For example, the blood substitute may be suitable for use with all blood types and may have an oxygen and/or carbon dioxide solubility higher than that of non-oxygenated saline solution.

Examples of suitable blood substitutes include oxygenated saline solution and OXYGENT™, which is the trademark for a blood substitute made by Alliance Pharmaceutical Corporation. OXYGENT™ is a perflubron emulsion; perflubron is a colorless, medical grade liquid perfluorochemical. At room temperature, perflubron has an oxygen solubility approximately 20 times that of non-oxygenated saline solution and a carbon dioxide solubility approximately 3 times that of non-oxygenated saline solution.

In various embodiments, the blood substitute may be continuously perfused into the vessel, which will reduce the refractory effects of the blood during imaging/treatment and the ischemic effects of a typical non-oxygenated flushing solution. Thus, if a blood substitute is used, timing may not be necessary. However, depending on the application, a blood substitute may be advantageously used in combination with the timing process described above.

Any of the features of the various embodiments disclosed herein may be used alone or in combination with other features of the various embodiments. For example, fluid dispersion devices may be included on a catheter that uses a timing mechanism to time flush introduction and moves the imaging device in a distal direction while imaging. Furthermore, balloons may be used to reduce the cross-sectional area of the vessel such that the amount of flush required may be reduced since only the reduced flow area of the vessel would require flushing.

Moreover, any of the various devices and methods may be automated. For example, insertion of the catheter, inflation of the balloon, movement of the imaging/treatment device while imaging/treating, introduction of the flush, etc., may all be automated.

It is to be understood that even though numerous characteristics and advantages of various embodiments have been set forth in the foregoing description, together with details of structure and function of the various embodiments, this disclosure is illustrative only. Changes may be made in detail, especially matters of structure and management of parts, without departing from the scope of the various embodiments as expressed by the broad general meaning of the terms of the appended claims.

We claim:

1. An apparatus comprising:
a cannula having a dimension suitable for insertion into a vessel of a subject, the cannula comprising a surface and defining a lumen, wherein the lumen is defined by at least one lumen opening to the outer surface of the cannula; and
a fluid dispersion device coupled to the cannula in a manner to disperse fluid introduced from the lumen opening into a flow of fluid within the vessel.

2. The apparatus of claim 1, wherein the fluid dispersion device is coupled to the cannula at a point proximal to the at least one lumen opening and the apparatus further comprises:
at least one protrusion disposed on an outer surface of the cannula at a point distal to the at least one lumen opening.

3. The apparatus of claim 2, wherein the fluid dispersion device is coupled to the cannula at a position proximal to the at least one lumen opening and extends distally beyond the at least one lumen opening and beyond a portion of the at least one protrusion.

4. The apparatus of claim 1, wherein the lumen is defined by a plurality of lumen openings.

5. The apparatus of claim 4, further comprising a plurality of protrusions, wherein respective ones of the plurality of fluid dispersion devices are disposed distal to a plurality of the lumen openings.

6. The apparatus of claim 4, wherein distal lumen openings are generally larger than proximal lumen openings.

7. The apparatus of claim 1, further comprising:
a timer to regulate introduction of an injectate from at least one lumen opening defined by the catheter into the flow of fluid in the vessel.

8. The apparatus of claim 7, wherein the timer may regulate introduction of the injectate at a predetermined portion of a cardiac cycle of the subject.

9. The apparatus of claim 7, wherein the timer may regulate introduction of the injectate for a predetermined amount of time.

10. The apparatus of claim 1, wherein the fluid dispersion device is an inflatable balloon located at a position distal to the at least one lumen opening and the at least one lumen opening comprises a lumen opening directed in a distal direction.

11. The apparatus of claim 1, wherein the fluid dispersion device is coupled to the cannula at a point proximal to the at least one lumen opening and comprises a shape-modifiable framework and a material coupled to the framework.

12. The apparatus of claim 11, wherein the shape-modifiable framework comprises a proximal end coupled to the cannula and a distal end comprising a diameter, in one position, greater than the diameter of the cannula.

13. The apparatus of claim 12, wherein the shape-modifiable framework comprises a material having a shape memory property to adopt the diameter greater than the diameter of the cannula when exposed to a condition within a blood vessel.

14. An apparatus comprising:
a catheter suitable for insertion into a vessel of a subject, the catheter including a balloon comprising a completely expanded position to selectively partially occlude a flow of fluid within the vessel; and
a light-emitting device to perform at least one of imaging and treating at least a portion of the vessel.

15. The apparatus of claim 14, wherein the light-emitting device comprises:
at least one of an optical coherence tomography device, an intravascular ultrasound device, and a photodynamic therapy device.

16. The apparatus of claim 14, wherein the balloon is inflatable.

17. The apparatus of claim 14, wherein the balloon defines at least one channel that is substantially parallel to a longitudinal axis of the catheter.

18. The apparatus of claim 17, wherein the channel is formed within at least one of an outer portion and an inner portion of the balloon.

19. The apparatus of claim 17, wherein the channel has dimensions to permit the fluid in the vessel to flow through the channel without completely degrading the ability of the light emitting device to one of image and treat at least a portion of the vessel.

20. The apparatus of claim 14, wherein the balloon is lobe-shaped and winds around at least a portion of the catheter.

21. The apparatus of claim 20, wherein the light-emitting device is disposed relative to the balloon such that the flow of fluid is at least one of redirected away from and reduced in an area in which the light-emitting device is positioned to one of image and treat.

22. The apparatus of claim 14, further comprising:
a timer to regulate introduction of an injectate from at least one lumen opening defined by the catheter into the flow of fluid in the vessel.

23. The apparatus of claim 22, wherein the timer may be controlled to regulate introduction of the injectate at a predetermined portion of a cardiac cycle of the subject.

24. The apparatus of claim 22, wherein the timer may regulate introduction of the injectate for a predetermined amount of time.

25. The apparatus of claim 14, further comprising a cannula disposed within the catheter, the cannula defining a lumen and at least one lumen opening through the catheter at a point proximal to the balloon.

26. The apparatus of claim 25, wherein the at least one lumen opening comprises a lumen opening directed in a distal direction.

27. A method comprising:
introducing a catheter into a vessel of a subject, the catheter comprising at least one of a fluid dispersion device and a balloon having an inflated state to partially occlude a flow of fluid within the vessel; and
timing the introduction of an injectate from at least one lumen opening defined by the catheter into a flow of fluid in the vessel.

28. The method of claim 27, wherein timing includes introducing the injectate at a predetermined portion of a cardiac cycle of the subject.

29. The method of claim 27, wherein timing includes introducing the injectate for a predetermined amount of time.

30. The method of claim 27, further comprising:
at least one of imaging and treating at least a portion of the vessel with a light-emitting device.

31. The method of claim 30, wherein timing includes introducing the injectate such that the injectate is disposed adjacent to the light-emitting device during a predetermined portion of a cardiac cycle of the subject.

32. The method of claim 27, wherein the injectable comprises an electromagnetic wave-transparent oxygen carrier.

33. A kit comprising:
a catheter suitable for insertion into a vessel of a subject and a cannula therethrough including a lumen opening to an outer surface of the catheter proximal to a distal end of the catheter; and
at least one of an imaging and treating device;
an injectate suitable for injection into the cannula.

34. The apparatus of claim 33, wherein the light-emitting device comprises one of an optical coherence tomography device, an intravascular ultrasound device, and a photodynamic therapy device.

35. The apparatus of claim 33, further comprising:
a timer to regulate introduction of an injectate from at least one lumen opening defined by the catheter into a flow of fluid in the vessel.

36. The apparatus of claim 35, further comprising:
a processor including instructions to regulate introduction of the injectate according to the timer.

37. A method comprising:
introducing a catheter into a vessel of a subject; and
at least one of imaging and treating at least a portion of the vessel by moving a device in a distal direction relative to a proximal section of the catheter.

38. The method of claim 37, further comprising:
introducing an injectate from at least one lumen opening defined by the catheter into a flow of fluid in the vessel such that the injectate travels in a same direction of travel of the light-emitting device during imaging and/or treatment.

39. The method of claim 37, wherein the injectate comprises a blood substitute.
